(19) United States
(12) Patent Application Publication
(10) Pub. No.: US 2024/0342376 A1
(43) Pub. Date: Oct. 17, 2024

(54) A slider and a sub-assembly for a medicament delivery device

(71) Applicant: SHL Medical AG, Zug (CH)

(72) Inventors: Ming-Ting Yin, Taoyuan City (TW); Philipp Zingel, Taoyuan City (TW); Roman Käslin, Zurich (CH); Lino Manuel Heckhorn Ghilardi, Zurich (CH)

(21) Appl. No.: 18/293,886

(22) PCT Filed: Aug. 3, 2022
(86) PCT No.: PCT/EP2022/071824

§ 371 Date: Jan. 31, 2024

(30) Foreign Application Priority Data
Aug. 9, 2021 (EP) 21190456.0

(51) Int. Cl. A61M 5/20; A61M 5/315

(52) U.S. Cl. CPC A61M 5/2033; A61M 5/3158; A61M 2005/2013

(57) ABSTRACT

A slider of a medicament delivery device power pack, the slider comprising: a body extending along a longitudinal axis between a first end and a second end; and a straight guide surface extending from a wall of the body in a direction transverse to the longitudinal axis, and extending from a first end to a second end in a direction that is angled relative to both the longitudinal axis and to a plane transverse to the longitudinal axis.

### CROSS-REFERENCE TO RELATED APPLICATIONS

The present application is a U.S. National Phase Application pursuant to 35 U.S.C. § 371 of International Application No. PCT/EP2022/071824 filed Aug. 3, 2022, which claims priority to EP patent application Ser. No. 21/190,456.0 filed Aug. 9, 2021. The entire disclosure contents of these applications are herewith incorporated by reference into the present application.

### TECHNICAL FIELD

The present disclosure generally relates to a slider and a sub-assembly for a medicament delivery device, and particularly to a slider with a straight guide surface and a sub-assembly that comprises a slider for releasably holding a plunger rod against a power source of the medicament delivery device.

### BACKGROUND

Medicament delivery devices such as autoinjectors often include a powerpack for automatically expelling contained medicament once users have triggered the medicament delivery devices. A medicament delivery device usually comprises multiple functions for multiple demands. For example, before expelling contained medicament, the medicament delivery device might need to expel contained air (often refers as “priming”); or the medicament delivery device might contain two medicament substance that requires to be mixed before expelling to a user. Those functions are usually performed by the powerpack.

It has been appreciated that solutions for providing a powerpack of a medicament delivery device for carrying out multiple functions; therefore, a less complex medicament delivery device could be advantageous.

### SUMMARY

The present disclosure is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

The first aspect of the present disclosure provides a sub-assembly for a medicament delivery device, the sub-assembly comprising: a housing, a slider, an actuator and a plunger rod; the slider, the actuator and the plunger rod are all at least partially arranged within the housing; the housing extends along a longitudinal axis between a proximal end and a distal end; and wherein the housing comprises a retaining ledge extending in a first transverse direction transverse to the longitudinal axis from a wall of the housing; the slider comprises a holding surface, a retaining surface, and an inclined surface; the holding surface is a distally directed surface; wherein the retaining surface is a proximally directed surface; and wherein the inclined surface extends from a first end to a second end in the direction that is angled relative to both the longitudinal axis and to a plane transverse to the longitudinal axis; the retaining surface of the slider is adjacent to the retaining ledge of the housing; the actuator is movable relative to the housing only in the direction of the longitudinal axis; the actuator comprises an actuating surface; and the plunger rod comprises a counter holding surface; the counter holding surface is a proximally directed surface; during use the actuating surface is movable along the inclined surface from the first end of the inclined surface to the second end of the inclined surface, so that the slider is linearly moved relative to the housing in a second transverse direction transverse to both the longitudinal axis and the first transverse direction between a holding position where the holding surface of the slider is engaged with the counter holding surface of the plunger rod and a released position where the holding surface of the slider is disengaged with the counter holding surface of the plunger rod.

Preferably, according to another embodiment, the inclined surface faces towards the proximal end of the housing; and the actuating surface faces towards the distal end of the housing.

Alternatively, according to another embodiment, the inclined surface faces towards the distal end of the housing; and the actuating surface faces towards the proximal end of the housing.

Preferably, according to another embodiment, the housing comprises a proximally directed surface adjacent to a second retaining surface of the slider.

Preferably, according to another embodiment, an interface formed between the retaining ledge of the housing and the retaining surface of the slider is perpendicular to the longitudinal axis.

Preferably, according to another embodiment, the actuator comprises a body and a protrusion extending from a wall of the body; and the actuating surface of the actuator is arranged on the protrusion.

Preferably, according to another embodiment, the slider comprises a transverse surface extending in the direction of the longitudinal axis and facing in the second transverse direction; and the protrusion of the actuator comprises a counter transverse surface that is configured to be adjacent to the transverse surface of the slider before the actuating surface contacts the first end of the inclined surface

Preferably, according to another embodiment, an interface formed between the holding surface of the slider and the counter holding surface of the plunger rod is perpendicular to the longitudinal axis.

Alternatively, according to another embodiment, an interface formed between the holding surface of the slider and the counter holding surface of the plunger rod is a ramp surface.

Preferably, according to another embodiment, the slider comprises a slider body extending along the longitudinal axis between a proximal end and a distal end.

Preferably, according to another embodiment, the slider comprises a straight ledge extending from a surface of the body in the first transverse direction to the longitudinal axis; and the inclined surface defined by the straight ledge.

Preferably, according to another embodiment, the slider comprises a counter retaining ledge extending from a surface of the body in the first transverse direction; and the retaining surface of the slider is defined by the retaining ledge.

Preferably, according to another embodiment, the second retaining surface of the slider is defined by the retaining ledge.

Preferably, according to another embodiment, the plunger rod comprises an elongated plunger rod body extending along the longitudinal axis between a proximal end and a distal end.

Preferably, according to another embodiment, the elongated plunger rod body of the plunger rod comprises an enlarged proximal part; the enlarged proximal part has a diameter greater than a diameter of the rest of the body; and the counter holding surface of the plunger rod is arranged on a surface of the enlarged portion of the body.

Preferably, according to another embodiment, the counter holding surface of the plunger rod comprises a first counter holding surface and a second counter holding surface; the first counter holding surface is contiguous with and axially offset relative to the second counter holding surface.

Preferably, according to another embodiment, the slider comprises a second inclined surface extending from the wall of the body in the first transverse direction to the longitudinal axis, and extending from a first end to a second end in a direction that is angled relative to both the longitudinal axis and to a plane transverse to the longitudinal axis; the actuator comprises a second actuating surface; and the second actuating surface is axially movable along the second inclined surface from the first end to the second end.

Preferably, according to another embodiment, the second actuating surface is the same surface as the actuating surface.

Preferably, according to another embodiment, when the second actuating surface is configured to move along the second inclined surface from the first end to the second end, the slider is configured to be linearly movable relative to the housing in the second transverse direction between an initial position where the holding surface of the slider is engaged with the first counter holding surface of the plunger rod and the holding position where the holding surface of the slider is disengaged with the second counter holding surface of the plunger rod.

Preferably, according to another embodiment, the housing comprises a housing body extending along a longitudinal axis between a proximal end and a distal end, and a flexible arm attached to the proximal end of the housing body.

Preferably, according to another embodiment, the flexible arm extends from the housing body towards the longitudinal axis.

Preferably, according to another embodiment, the flexible arm comprises a pair of arms extending from the housing body towards the longitudinal axis; a central passage extending along the longitudinal axis is defined between the pair of arms.

Preferably, according to another embodiment, the medicament delivery device is an injection device, an inhalation device, or a medical sprayer.

Preferably, according to another embodiment, when the medicament delivery device is an injection device, the medicament delivery device is an auto-injector.

The second aspect of the present disclosure provides a slider of a medicament delivery device power pack, the slider comprising: a body extending along a longitudinal axis between a first end and a second end; and a straight guide surface extending from a wall of the body in the first transverse direction, and extending from a first end to a second end in a direction that is angled relative to both the longitudinal axis and to a plane transverse to the longitudinal axis.

Preferably, according to another embodiment, a cut-out or recess is arranged in the wall of the body; and the straight guide surface is an edge of the cut-out or recess.

Alternatively, according to another embodiment, the slider a straight ledge extending from the wall of the body in the first transverse direction; and the straight guide surface is arranged on the straight ledge.

The second aspect of the present disclosure provides a sub-assembly for a medicament delivery device, the sub-assembly comprising: a housing extending along a longitudinal axis between a proximal end and a distal end; a plunger rod arranged within the housing; a slider configured to releasably engage with the plunger rod; and an actuator at least partially arranged within the housing and axially movable relative to the slider; the slider is linearly movable relative to the housing in the second transverse direction between a holding position where the plunger rod is engaged with the slider and a release position where the plunger rod is disengaged with the slider; the slider comprises a body and a track arranged on the body; the actuator comprises a track follower configured to move along the guide track of the slider when the actuator axially moves relative to the slider from the first position to the second position, thereby moves the slider from the holding position to the release position.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1A schematically shows an exploded view of a sub-assembly of a medicament delivery device.

FIG. 1B schematically shows a perspective view of the sub-assembly of FIG. 1A.

FIG. 1C schematically shows a perspective view of a plunger rod of the sub-assembly of FIG. 1A.

FIG. 1D schematically shows a perspective view of the plunger rod and a slider of the sub-assembly of FIG. 1A.

FIG. 2 schematically shows a perspective view of the plunger rod of FIG. 1C and a plunger rod spring.

FIG. 3A schematically shows a side view of the slider of FIG. 1D.

FIG. 3B schematically shows a perspective view of an actuator of the sub-assembly of FIG. 1A.

FIGS. 4A-4B schematically show perspective views of the slider of FIG. 1D and a housing of the sub-assembly of FIG. 1A.

FIG. 4C schematically shows a perspective view of the slider of FIG. 1D and the plunger rod of FIG. 1D.

FIG. 5 schematically shows a cross-section view of an indication mechanism used with the sub-assembly of FIG. 1A.

FIG. 6A schematically shows a perspective view of an indication sleeve of the indication mechanism of FIG. 5.

FIG. 6B schematically shows a perspective view of the indication sleeve of the indication mechanism of FIG. 6A.

FIG. 6C schematically shows the indication sleeve of the indication mechanism of FIG. 6A with an extension.

FIG. 7A schematically shows a perspective view of a guide rod of the indication mechanism of FIG. 5.

FIG. 7B schematically shows a cross-section view of the guide rod of FIG. 7A with the indication sleeve of the indication mechanism of FIG. 6A.

FIG. 7C schematically shows the plunger rod of FIG. 1D with an interaction portion for interacting with the indication mechanism of FIG. 5.

### DETAILED DESCRIPTION

A first aspect of the present disclosure, illustrated by FIG. 3A, discloses a slider 2 of a medicament delivery device power pack. The slider 2 is configured to releasably hold a plunger rod 4 of the medicament delivery device against a force from a power source. The slider 2 is configured to interact with a trigger of the medicament delivery device, so that a movement of the trigger of the medicament delivery device moves the slider 2 to disengage with the plunger rod 4. The slider 2 comprises a body 20 extending along a longitudinal axis L′ between a first end and a second end; and a straight guide surface 22 extending from a wall of the body 20 in a first transverse direction transverse to the longitudinal axis L′, and extending from a first end 22a to a second end 22b in the direction that is angled relative to both the longitudinal axis L′ and to a plane transverse to the longitudinal axis L′. The angle between the straight guide surface 22 and the longitudinal axis L′; and the angle between the straight guide surface 22 and the plane transverse to the longitudinal axis L′ can be 10-80 degrees, preferably 30-70 degrees, dependent on the design of the medicament delivery device.

The slider 2 therefore can move in a second transverse direction transvers to both the longitudinal axis L′ and the first transverse direction when the slider 2 is used in a medicament delivery device. The first transverse direction means the direction of a first transverse axis, and the second transverse direction means the direction of a second transverse axis. The first transverse axis is perpendicular to the longitudinal axis L; and the second transverse axis is perpendicular to an plane defined by the longitudinal axis and the first transverse axis.

In one example, a cut-out or recess is arranged in the wall of the body; and the straight guide surface is an edge of the cut-out or recess. In another example, a straight ledge extends from the wall of the body in the first transverse direction; and the straight guide surface is arranged on the straight ledge.

When the slider 2 is used with a medicament delivery device power pack, the trigger of the medicament delivery device is movable only in the axial direction relative to the housing of the medicament delivery device. The trigger of the medicament delivery device comprises an interacting member configured to move along the straight guide surface 22 of the slider 2, so that the slider 2 is moved linearly by the trigger of the medicament delivery device in the second transverse direction.

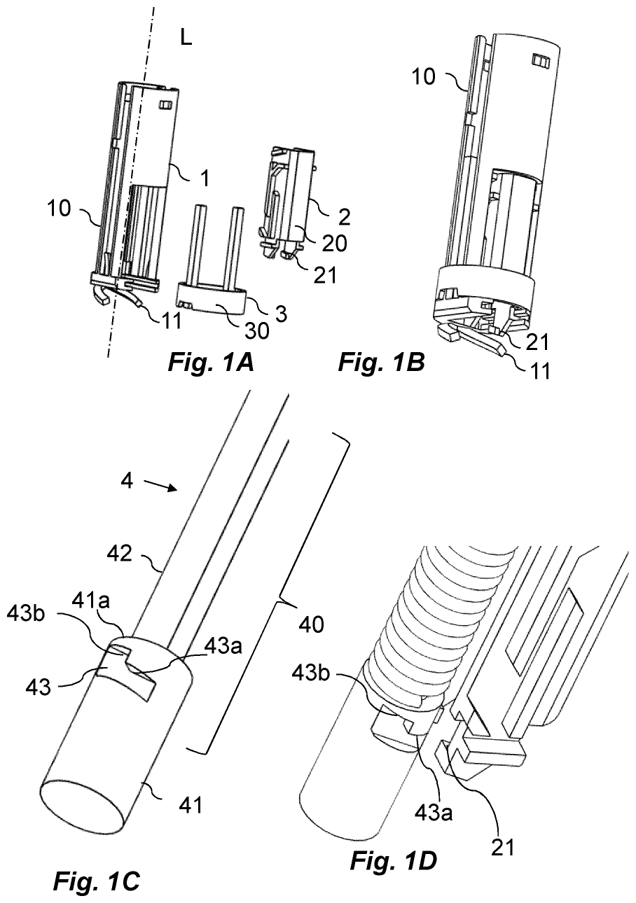

Furthermore, FIGS. 1-7C illustrate a second aspect of the present disclosure, a sub-assembly of a medicament delivery device. The sub-assembly comprises the slider 2, for example as mentioned above. The sub-assembly comprises a housing 1 extending along a longitudinal axis L between a proximal end and a distal end, the slider 2, an actuator 3 and a plunger rod 4 configured to expel a contained medicament of the medicament delivery device under a force from a power source of the medicament delivery device. The power source can be: a spring, e.g. compression spring, tension spring, or torsion spring; or a gas canister or a motor gear, dependent on the design of the medicament delivery device. When the slider 2 is assembled to the sub-assembly as described below, the longitudinal axis L′ of the slider 2 is the same as the longitudinal axis L of the housing 1, or is parallel to the longitudinal axis L of the housing 1.

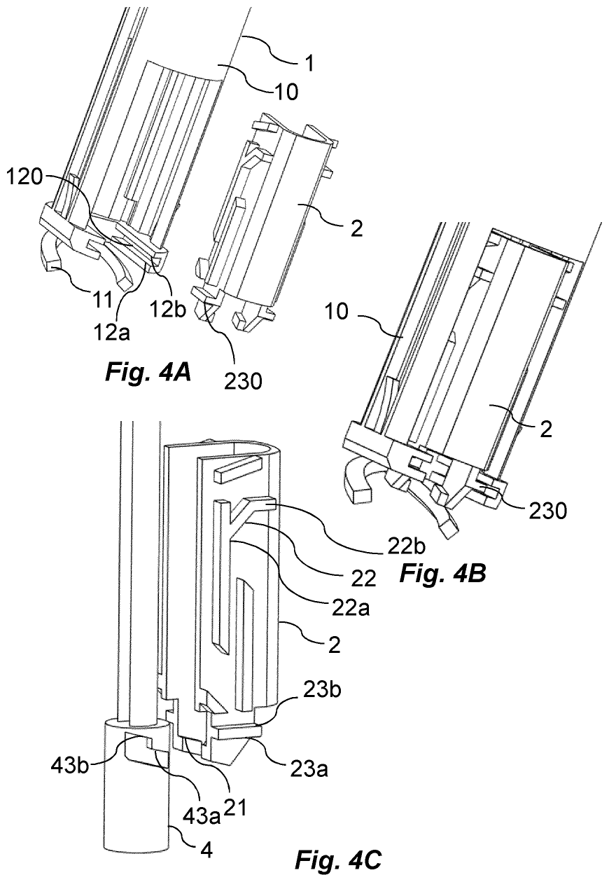

The housing 1 comprises a housing body 10 extending along the longitudinal axis between a proximal end and a distal end, a retaining ledge 12a, optionally a proximally directed surface 12b, and optionally a flexible arm 11 extending from the proximal end of the housing body 10, as shown in FIG. 1A and FIG. 4A. The housing body 10 is configured to accommodate the slider 2, the actuator 3, and the plunger rod 4. The housing body 10 can be formed in a generally tubular shape, or any other suitable shape dependent on the shape of the medicament delivery device comprising the sub-assembly of the present disclosure. In one example, the retaining ledge is arranged at the proximal end of the housing body 10; and the proximally directed surface 12b is arranged at the distal end of the housing body 10. For example, the retaining ledge protrudes from the housing body 10 at its proximal end in the first transverse direction; the proximally directed surface can be defined by a ledge protruding from the housing body 10 at its distal end in the first transverse direction. In another example, the retaining ledge 12a and the proximally directed surface 12b define a groove or cut-out 120 at the proximal end of the housing body 10, as shown in FIG. 4A. In a preferred example, the flexible arm 11 of the housing 1 is configured to abut a medicament container of the medicament delivery device at a distal end of the medicament container, so that an engineering tolerance of the length of the medicament container can be tolerated by the flexible arm 11, and/or the medicament container of the medicament delivery device can be blocked from axially moving in the distal direction. Furthermore, the flexible arm 11 can also absorb a shock that is transmitted to the distal end of the medicament container of the medicament delivery device, so that damage of the medicament container of the medicament delivery device can be prevented.

The slider 2 is configured to releasably engage with the plunger rod 4 to hold back the force from the power source of the medicament delivery device. The slider 2 comprises the body 20, a holding surface 21, a retaining surface 23a, a second retaining surface 23b and the straight guide surface 22. The holding surface 21 is a distally directed surface. The retaining surface 23a is a proximally directed surface. The second retaining surface 23b is a distally directed surface. The straight guide surface 22 is an inclined surface that extends from a first end 22a to a second end 22b in the direction that is angled relative to both the longitudinal axis L and to a plane transverse to the longitudinal axis L. The slider body 20 can be formed in any suitable shape such as a planar or two planar connected by a joint; a planar rectangular component or two planar rectangular components connected by a joint. The slider 2 may further comprises a proximal extension extending from the planar part in the proximal direction of the housing 1, or the planar rectangular part in the proximal direction of the housing 1, as shown in FIG. 3A. In one example, the slider body 20 comprises a wall with multiple ledges extending from the wall in the first transverse direction; and/or multiple recesses or cut-outs extending in the first transverse direction. In one example, the inclined surface 22 is defined by a straight ledge 220 of the slider body 20, as shown in FIG. 1D and FIG. 3A.

The slider 2 is linearly movable relative to the housing 1 in the second transverse direction. The slider 2 is engaged with the housing 1 through an engagement between the retaining surface 23a and the retaining ledge 12a and an engagement between the second retaining surface 23b and the proximally directed surface 12b. Preferably, the linear movement of the slider is along an interface between the retaining surface 23a and the retaining ledge 12a and an interface between the second retaining surface 23b and the proximally directed surface 12b.

It should be noted that the engagement between the second retaining surface 23b and the proximally directed surface 12b can be replaced by an engagement between the second retaining surface 23b and a proximally directed surface of a component of the medicament delivery device that is attached to the housing 1, e.g. a distal cap. In a preferred example, the interface formed between the retaining ledge 12a of the housing 1 and the retaining surface 23a of the slider 2 is perpendicular to the longitudinal axis L. In this example, a space for the slider 2 to move could be minimized, therefore, the housing 1 can be more compact. Furthermore, because the force from the power source will transfer to the slider 2 through an interface between the holding surface 21 of the slider 2 and the counter holding surface 43a, 43b of the plunger rod 4, the slider 2 is therefore difficult to move in the distal direction relative to the housing 1. Therefore, if the interface formed between the retaining ledge 12a of the housing 1 and the retaining surface 23a of the slider 2 is perpendicular to the longitudinal axis L, the slider 2 is generally axially fixed to the housing 1 in the axial direction.

In another preferred example, the retaining surface 23a and the second retaining surface 23b are defined by a counter retaining ledge 230, as shown in FIG. 3A. In this example, the counter retaining ledge 230 is configured to be positioned within the groove 120 of the housing, as shown in FIGS. 4A-4B. Optionally, the slider 2 may comprise a transverse surface 24, a second holding surface, and a second inclined surface 25. The transverse surface 24 extends in the direction of the longitudinal axis L and faces in second transverse direction. The transverse surface 24 faces in the direction that the slider is also movable in. The second holding surface is contiguous and axially offset relative to the holding surface of the slider. The second inclined surface 25 extends from the wall of the body 20 in the first transverse direction, and extending from a first end 25a to a second end 25b in a direction that is angled relative to both the longitudinal axis L and to a plane transverse to the longitudinal axis. The angle between the second inclined surface 25 and the longitudinal axis L; and the angle between the second inclined surface 22 and the plane transverse to the longitudinal axis can be 10-80 degrees, preferably 30-70 degrees, dependent on the design of the medicament delivery device. The transverse surface 24, the second holding surface, and the second inclined surface 25 are defined by the ledges, as shown in FIG. 3A, and/or cut-outs of the wall of the body 20 of the slider 2. Furthermore, in another example, the slider 2 optionally comprises one or more extra proximally directed surfaces 26; similarly, the one or more extra proximally directed surfaces 26 can be defined by one or more ledges extending from the slider body 20 in the first transverse direction; or can be defined by one or more edges of cut-outs or recesses on the slider body 20. The extra proximally directed surfaces 26 can be used to releasably hold one or more extra functional components of the medicament delivery device. For example, the medicament delivery device may comprise a distally biased indicator. In this example, the extra proximally directed surface 26 of the slider 2 may be used to releasably hold the distally biased indicator. Therefore, when the slider 2 slides relative to the housing 1 in second transverse direction from a holding position to a released position (will be explained in detail later), the distally biased indicator can be released and move in the distal direction of the housing to provide an indication, e.g. a visual, audible, and/or tactile indication; and/or switch on a switch for data transmission.

As shown in FIG. 3B, the actuator 3 comprises an actuator body 30, an actuating surface 31, an optional counter transverse surface 32, and an optional second actuating surface 33. The actuator 3 is at least partially arranged within the housing 1, and is fixed to the housing in the first and the second transverse directions, namely, the actuator 3 can only move relative to the housing 1 along the longitudinal axis L.

The actuator body 30 can be configured to connect to a user-accessible trigger of the medicament delivery device. The user-accessible trigger of the medicament delivery device can be a delivery member guard or a button that is configured to start the medicament delivery operation upon being axially moved by the user relative to the housing 1. The actuator 3 is configured to be axially moved by the user along the longitudinal axis L relative to the housing 1 and the slider 2. The actuator body 30 can be formed in any suitable shape depending on the shape of the medicament delivery device and/or dependent on the shape of the user-accessible trigger of the medicament delivery device. For example, a delivery member guard or a button of the medicament delivery device is often formed with a tubular portion, so that the actuator body 30 can be formed as a ring shape or tubular shape. Alternatively, the actuator 3 can act as a delivery member guard or a button of the medicament delivery device. In this example, the sub-assembly comprises a delivery member guard and/or a button for the medicament delivery device. In an example where the actuator is also the delivery member guard, the actuator body is elongated and comprises a tubular portion at its proximal end to cover a medicament delivery member. In an example where the actuator is the button of the delivery member guard, the actuator body comprises a finger pad so that the user can move the actuator body via the finger pad.

For being fixed to the housing in the first and the second transverse directions, the actuator body 3 may comprise a slot or a rib to interact with a counter rib on an inner surface of the housing body; or the actuator body may be formed with a shape matching with a structure on the inner surface of the housing body, for example, the actuator body is snugly fitted to the housing body, so that the actuator body cannot move relative to the housing body in the first and the second transverse directions. Alternatively, the actuator body 30 comprises a distally extending arm configured to be positioned within a slot on the housing body 10.

The inclined surface 22 acts as a guide track, and the actuating surface acts as a track follower. When the user axially moves the actuator 3 for triggering a medicament delivery operation (a trigger movement of the actuator 3), the actuating surface 31 of the actuator 3, along with the axial movement of actuator 3, travels along the inclined surface 22 from the first end 22a of the inclined surface 22 to the second end 22b of the inclined surface 22. The actuating surface 31 of the actuator 3 can be defined by a protrusion 310 extending from a surface of the actuator body 30 in the first transverse direction. Alternatively, the actuating surface of the actuator can be defined by a tip end of an arm extending from the actuator body 30 along the longitudinal axis L. When the actuating surface 31 of the actuator 3 travels along the inclined surface 22 from the first end 22a of the inclined surface 22 to the second end 22b of the inclined surface 22, the slider 2 is slid relative to the housing 1 in the second transverse direction from the holding position to the released position.

It should be noted that whether the actuating surface 31 and the inclined surface 22 should face towards the proximal end or the distal end of the housing 10 is dependent on the design of the medicament delivery device. For example, if the medicament delivery device is triggered by the movement of a delivery member guard, the actuating surface 31 faces the distal end of the housing 10 and the inclined surface 22 faces the proximal end of the housing 10. On the other hand, if the medicament delivery device is triggered by the movement of a button, the actuating surface faces the proximal end of the housing and the inclined surface faces the distal end of the housing.

The counter transverse surface 32 of the actuator 3 is configured to travel, along with the axial movement of the actuator 3, along the transverse surface 24 of the slider 2 before the actuating surface 31 reaches the first end 22a of the inclined surface 22. More detail about the transverse surface 22 of the slider and the counter transverse surface 32 of the actuator 3 will be provided later.

The second actuating surface 33 of the actuator 3 is configured to travel along the second inclined surface 25 of the slider 2 from the first end 25a of the inclined surface 25 to the second end 25b of the inclined surface 25. The second actuating surface 33 travels along the second inclined surface 25 together with an axial movement of the actuator 3 that has an opposite moving direction to the trigger movement of the actuator 3. When the second actuating surface 33 of the actuator 3 travels along the second inclined surface 25 from the first end 25a of the second inclined surface 25 to the second end 25b of the second inclined surface 25, the slider 2 is slid relative to the housing 1 in the second transverse direction between an initial position and the holding position.

Similarly, the counter transverse surface 32 of the actuator 3 can be defined by a protrusion 310 extending from a surface of the actuator body 30 in the first transverse direction. Alternatively, the counter transverse surface of the actuator can be defined by a sidewall of an arm extending from the actuator body 30 along the longitudinal axis L. The second actuating surface of the actuator 3 can be defined by a protrusion 310 extending from a surface of the actuator body 30 in first transverse direction. Alternatively, the second actuating surface of the actuator can be defined by a tip end of an arm extending from the actuator body 30 along the longitudinal axis L.

The plunger rod 4 comprises an elongated plunger rod body 40 and a counter holding surface 43a, 43b, as shown in FIGS. 1C-1D and FIG. 4C. The counter holding surface 43a, 43b of the plunger rod 4 is adjacent to the holding surface 21 of the slider 2 when the slider 2 is in the holding position. When the slider 2 is slid from the holding position to the released position by the actuator 3, the holding surface 21 is slid and therefore misaligned with the counter holding surface 43a, 43b of the plunger rod 4, so that the plunger rod 4 can be moved by the power source to expel the contained medicament of the medicament delivery device. The counter holding surface 43a, 43b of the plunger rod 4 can be defined by an edge of a recess/cut-out 43 on the elongated plunger rod body 40, as shown in FIGS. 1C-1D; or can be defined by a ledge protruding from the elongated plunger rod body 40 in first transverse direction.

In one example, an interface formed between the counter holding surface of the plunger rod 4 and the holding surface of the slider 2 is perpendicular to the longitudinal axis L. In this example, the plunger rod 4 can be driven to move in the proximal direction with rotation or without rotation by the power source. For example, if the plunger rod is driven to move in the proximal direction with rotation, the plunger rod comprises a screw or helical structure to convert the rotation of the plunger rod into an axial movement of the plunger rod. In this example, the interface formed between the counter holding surface of the plunger rod 4 and the holding surface of the slider 2 is perpendicular to the longitudinal axis L, so that the axial movement of the plunger rod will be blocked by the interface; therefore, the plunger rod cannot pass the screw or helical structure. On the other hand, if the plunger rod 4 is driven to move in the proximal direction without rotation, the perpendicular interface formed between the counter holding surface of the plunger rod 4 and the holding surface of the slider 2 will block the axial movement of the plunger rod 4.

In another example, the interface formed between the counter holding surface of the plunger rod 4 and the holding surface of the slider 2 is a ramp surface; or one of the counter holding surface of the plunger rod 4 and the holding surface of the slider 2 is a ramp surface. This example will be more suitable for the plunger rod 4 that is driven in the proximal direction without rotation. The axial force received by the plunger rod 4 from the power source will also transfer to the ramp surface, and therefore provides a transverse force to the slider 2. The slider 2 can therefore be slid to the released position with less force from the actuator 3. In this example, the engagement between the transverse surface 24 of the slider 2 and the counter transverse surface 32 of the actuator 3 is configured to additionally block the slider 2 from sliding in the second transverse direction relative to the housing 10.

Furthermore, in one example, the counter holding surface 43a, 43b of the plunger rod 4 comprises a first counter holding surface 43a, and a second counter holding surface 43b contiguous and axially offset relative to the first counter holding surface 43a, as shown in FIG. 1C and FIG. 4C; and/or the slider 2 comprises the second holding surface. For example, the first counter holding surface 43a of the plunger rod 4 engages with the holding surface 21 of the slider 2 when the slider 2 is in the initial position; on the other hand, the second counter holding surface 43b of the plunger rod 4 is spaced apart from the holding surface 21 of the slider 2 when the slider 2 is in the initial position. The first counter holding surface 43a of the plunger rod 4 is spaced apart from the holding surface 21 of the slider 2 when the slider 2 is in the holding position; on the other hand, the second counter holding surface 43b of the plunger rod 4 engages with the holding surface 21 of the slider 2 when the slider 2 is in the holding position.

In this example, when the slider 2 is linearly moved relative to the housing 1 in the second transverse direction between from the initial position to the holding position by the actuator 3, the plunger rod 4 can move in the proximal direction with an initial moving distance. The initial moving distance can be determined by an axial distance between the first counter holding surface 43a of the plunger rod 4 and the second counter holding surface 43b of the plunger rod 4. Alternatively, the initial moving distance can be determined by an axial distance between the holding surface 21 of the slider 2 and the second holding surface of the slider.

An interface formed between the first counter holding surface 43a of the plunger rod 4 and the holding surface 21 of the slider 2; and/or an interface formed between the second counter holding surface 43b of the plunger rod 4 and the holding surface 21 of the slider 2 can be a ramp surface or perpendicular to the longitudinal axis L; similarly, the second holding surface of the slider can be a ramp surface or perpendicular to the longitudinal axis L.

Alternatively, in one example the interface formed between the counter holding surface of the plunger rod 4 and the holding surface of the slider 2 is the ramp surface; or one of the counter holding surface of the plunger rod 4 and the holding surface of the slider 2 is a ramp surface. The plunger rod 4 may be released to move the initial moving distance when the slider 2 is linearly moved relative to the housing 1 in the second transverse direction between the initial position and the holding position by the actuator 3.

The initial moving distance can be used to perform an extra delivery operation of the medicament delivery device, e.g. expelling contained air in the medicament container and/or mixing two substances within the medicament container. The exact initial moving distance is dependent on the design of the medicament delivery device.

The slide of the slider 2 in the second transverse direction is coherent with either the extra delivery operation of the medicament delivery device or the medicament delivery operation of the medicament delivery device. Therefore, the one or more extra proximally directed surfaces 26 of the slider 2 can be used to releasably hold one or more components of the medicament delivery device that are performing a relevant function to the extra delivery operation of the medicament delivery device or the medicament delivery operation of the medicament delivery device. In the above-mentioned example, the indication of the medicament delivery device can be used to either provide an indication of the extra delivery operation of the medicament delivery device (indicates the medicament delivery device is ready for delivering the contained medicament); or an indication of the medicament delivery operation of the medicament delivery device (indicates the medicament delivery operation is started).

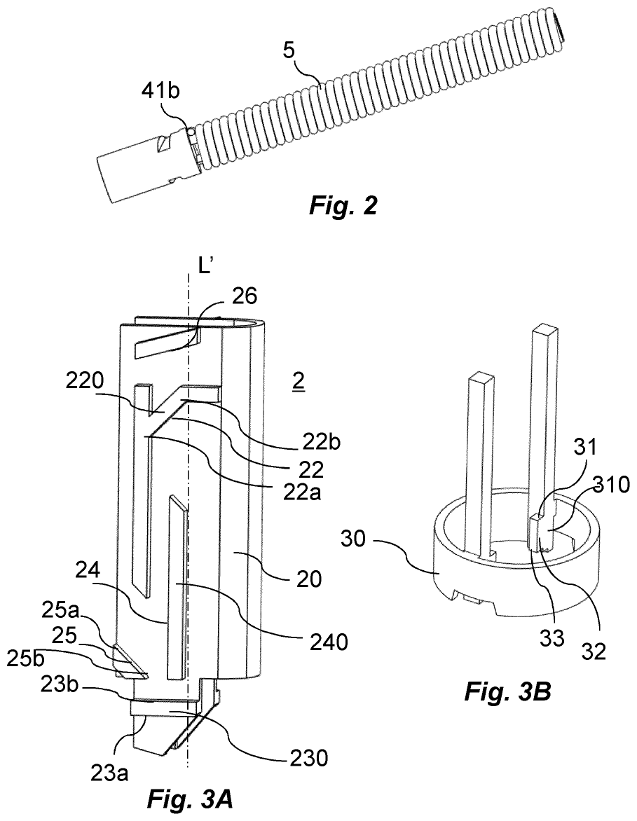

In another example, as shown in FIG. 1C and FIG. 2, the elongated plunger rod body 40 optionally comprises an enlarged proximal part 41 and a distal part 42. The enlarged proximal part 41 of the plunger rod 4 has a length measured in the first transverse direction being greater than a length of the distal part 42 measured in the first transverse direction. Therefore, a distally directed surface 41b is formed at the distal end of the enlarged proximal part 41 of the plunger rod 4. The plunger rod 4 as described in this example is suitable for being used with a compression spring 5 as the power source of the medicament delivery device. The compression spring 5 is configured to be adjacent to the distally directed surface 41a of the plunger rod 4 at one end, and adjacent to a wall of the housing 1, or other components that cannot move relative to the housing 1 before the medicament delivery operation started, at the other end. In this example, the enlarged proximal part 41 of the plunger rod 4 is configured to evenly distribute the force on a stopper of the medicament container of the medicament delivery device, so that a width of the enlarged proximal part 41 of the plunger rod 4 (the length measured in the first transverse direction) should be similar to a width of the stopper of the medicament container. On the other hand, the distal part 42 of the plunger rod 4 is configured to support the compression spring 5 from buckling, so the width of the distal part 42 of the plunger rod 4 only needs to allow the distal part 42 to snugly fit within an interior space of the compression spring 5. In this example, the counter holding surface 43a, 43b of the plunger rod 4 is arranged on the enlarged proximal part 41 of the plunger rod, as shown in FIG. 1C.

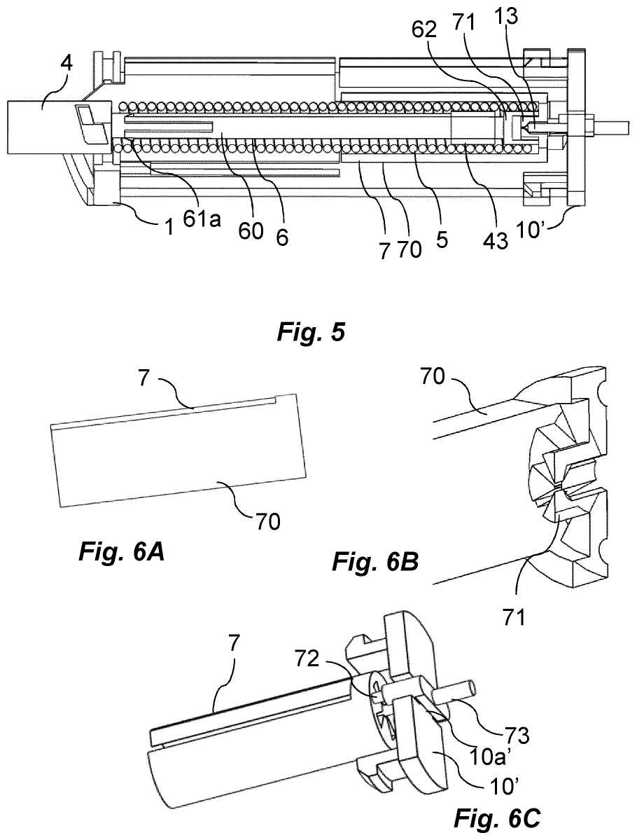

In another example, the sub-assembly of the present disclosure can be used with an indication mechanism for the medicament delivery device. As shown in FIGS. 5-7C, the indication mechanism comprises a guide rod 6 and an indicator 7. For being used with the indication mechanism, the housing 1 of the sub-assembly or an outer shell of the medicament delivery device comprises a distal wall 10′ and a protrusion 13 extending from the distal wall 10′ along the longitudinal axis L in the proximal direction.

The indicator 7 comprises an indicator body 70 extending between a proximal end and a distal end, and a plurality of arms 71 extending from the distal end of the indicator body 70 in the proximal direction. The plurality of flexible arms 71 each comprise ledges extending in the first transverse direction from proximal ends of the plurality of flexible arms 71. The indication mechanism is suitable for a medicament delivery device with a compression spring 5 as the power source. The compression spring 5 is adjacent to a plunger rod of the medicament delivery device at one end and adjacent to a distal wall of the indicator body at the other end. When the indication mechanism is used with the sub-assembly of the present disclosure, the compression spring 5 is adjacent to the distally directed surface 41a of the plunger rod 4 at one end, and adjacent to the distal wall of the indicator body 70 of the indicator 7 at the other end. The indicator 7 is configured to be biased by the compression spring 5 from a start position to an indicated position. The plurality of flexible arms 71 surround the protrusion 13 of the housing 1 or the outer shell of the medicament delivery device when the indicator 7 is in the start position.

Optionally, the indicator 7 comprises an extension 72 extending from the distal end of the indicator body 70 in the distal direction. Optionally, the extension 72 comprises a further protruding part 73 configured to protrude out from the distal wall 10′ of the housing 1 of the sub-assembly of the present disclosure, or from the distal wall of the outer shell of the medicament delivery device when the indicator 7 is in the indicated position. In this example, the distal wall 10′ comprises an opening 10a′ for allowing the protruding part 73 to pass. The protruding part 73 can therefore provide a visual and a tactile indication to the user when the indicator 7 is in the indicated position. On the other hand, if the medicament delivery device is used with an add-on device, such as an information tracking device that attaches to the medicament delivery device, the protruding part 73 can either switch on a switch of the add-on device or act as a detectable object for being detected by the add-on device.

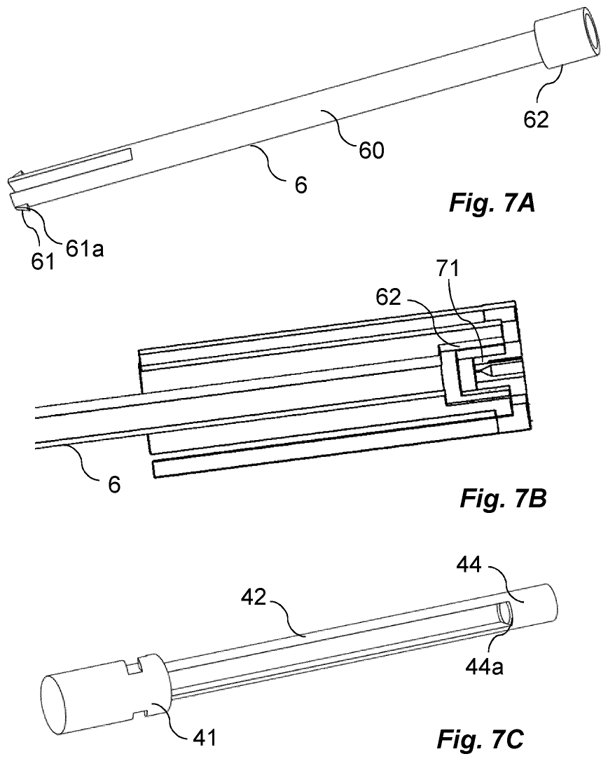

The guide rod 6 of the indication mechanism comprises an elongated guide rod body 60 extending along the longitudinal axis L between a proximal end and a distal end, a distally directed surface 61a, and a tubular portion 62, as shown in FIG. 7A. The elongated guide rod body 60 is at least partially received within a portion of the plunger rod of the medicament delivery device. For example, when the indication mechanism is used with the sub-assembly of the present disclosure, the plunger rod 4 of the sub-assembly comprises a proximally directed surface 44a. The proximally directed surface 44a can be defined by a ledge 44 extending from the plunger rod body towards the elongated guide rod body 60. The ledge 44 can be an annular ledge. The plunger rod is axially movable relative to the guide rod 6 between a first position and a second position.

The distally directed surface 61a of the guide rod 6 can be defined by a protrusion 61 extending from the elongated guide rod body 60 in the first transverse direction. Alternatively, the distally directed surface 61a of the guide rod 6 can be defined by an edge of a cut-out/recess arranged on the elongated guide rod body 60. The distally directed surface 61a of the guide rod 6 is spaced apart from the proximally directed surface 44a of the plunger rod when the indicator 7 is in the first position of the plunger rod; and the distally directed surface 61a of the guide rod 6 is adjacent to the proximally directed surface 44a of the plunger rod when the indicator 7 is in the second position of the plunger rod.

The tubular portion 62 of the guide rod 6 is configured to at least partially surround the plurality of flexible arms 71 of the indicator 7 when the indicator 7 is in the start position, as shown in FIG. 7B. Therefore, the plurality of flexible arms 71 of the indicator 7 cannot flex radially outward relative to the longitudinal axis L when the indicator 7 is in the start position. The plurality of flexible arms 71 of the indicator, at the same time, surround the protrusion 13 of the housing 1 or the outer shell of the medicament delivery device, the proximal end of the protrusion 13, therefore, is adjacent to the ledges of the plurality of flexible arms 71. An engagement between the proximal end of the protrusion 13 and the ledges of the plurality of flexible arms 71 thus stops the indicator 7 from being moved by the compression spring 5 in the distal direction relative to the distal wall 10′ of the housing 1 or the outer shell of the medicament delivery device.

In one example, the indication mechanism can be used to indicate the end of the medicament delivery operation. In this example, the distally directed surface 61a of the guide rod 6 is arranged at the proximal end of the elongated guide rod body 60 of the guide rod 6. When the medicament delivery operation starts, the plunger rod moves from the first position towards the second position under the force of the compression spring 5. Therefore, the proximally directed surface 44a of the plunger rod moves towards the distally directed surface 61a of the guide rod 6. When the plunger rod reaches the second position, the proximally directed surface 44a of the plunger rod engages with the distally directed surface 61a of the guide rod 6.

Once the proximally directed surface 44a of the plunger rod engages with the distally directed surface 61a of the guide rod 6, the guide rod 6 will be pulled by the plunger rod, under the force of the compression spring 5, in the proximal direction relative to the housing or the outer shell of the medicament delivery device. When the tubular portion 62 of the guide rod 6 moves away from surrounding the plurality of flexible arms 71 of the indicator 7, the flexible arms 71 of the indicator 7 flex radially outwardly relative to the protrusion 13 of the housing or the outer shell of the medicament delivery device. The ledges of the flexible arms 71 thus no longer engage with the protrusion 13 of the housing or the outer shell of the medicament delivery device, and the indicator 7 will then move towards the indicated position under the force of the compression spring 5. In one example, an audible indication can be generated when the indicator body 70 hits on the distal wall 10′ of the housing or the outer shell of the medicament delivery device, and/or a visual indication can be provided by having at least a part of the indicator body 70 be observable from a transparent portion or an opening of the medicament delivery device.

The indication mechanism can also be arranged as a start indication for the medicament delivery operation, and it can be done by positioning the proximally directed surface 44a of the plunger rod closer to the distally directed surface 61a of the guide rod 6, meaning the guide rod 6 can be shorter or the distally directed surface 61a of the guide rod 6 might be arranged closer to the distal end of the elongated guide rod body 60; or the proximally directed surface 44a of the plunger rod can be arranged closer to the proximal end of the plunger rod. The exact position of the proximally directed surface 44a of the plunger rod relative to the distally directed surface 61a of the guide rod 6 is dependent on the design of the medicament delivery device, e.g. end indication or start indication, and/or a delivery volume of the medicament.

It should be noted that, the term “perpendicular” in this description for describing the retaining surface, the interface(s), and/or the holding surface, is used to describing the mechanical structure that can use one of those mentioned surfaces to keep anther component (counter component), by engaging the a surface of the component, from moving in the direction of the longitudinal axis L, in at least the proximal direction or the distal direction relative to the housing 1. Therefore, not only when those surfaces are 90 degree angled relative to the longitudinal axis L can perform the holding function as described above, those surfaces can be, for example, around 80-110 degrees relative to the longitudinal axis L, or any other angle that can still keep the counter component immovable in the direction of the longitudinal axis L, in at least the proximal direction or the distal direction relative to the housing 1. Therefore, those angle between the retaining surface and the longitudinal axis L should be all fell within the meaning of “perpendicular” here.

The inventive concept has mainly been described above with reference to a few examples. However, as is readily appreciated by a person skilled in the art, other embodiments than the ones disclosed above are equally possible within the scope of the inventive concept, as defined by the appended claims.

1-14. (canceled)

15. A sub-assembly for a medicament delivery device, the sub-assembly comprising:
a housing;
a slider;
an actuator;
and a plunger rod,
wherein the slider, the actuator, and the plunger rod are all at least partially arranged within the housing,
wherein the housing extends along a longitudinal axis between a proximal end and a distal end,
wherein the housing comprises a retaining ledge extending in a first transverse direction transverse to the longitudinal axis from a wall of the housing,
wherein the slider comprises a holding surface and a retaining surface,
wherein the holding surface is a distally directed surface,
wherein the retaining surface is a proximally directed surface,
wherein the retaining surface of the slider is adjacent to the retaining ledge of the housing,
wherein the actuator is movable relative to the housing only in the direction of the longitudinal axis,
wherein the plunger rod comprises a counter holding surface,
wherein the counter holding surface is a proximally directed surface,
wherein the during use the actuator is configured to move the slider, so that the slider is linearly moved relative to the housing in a second transverse direction transverse to both the longitudinal axis and the first transverse direction between a holding position where the holding surface of the slider is engaged with the counter holding surface of the plunger rod and a released position where the holding surface of the slider is disengaged with the counter holding surface of the plunger rod.

16. The sub-assembly of the medicament delivery device according to claim 15, wherein the slider comprises an inclined surface, wherein the actuator comprises an actuating surface, wherein during use the actuating surface is movable along the inclined surface from the first end of the inclined surface to the second end of the inclined surface, so that the slider is linearly moved relative to the housing in the second transverse direction transverse to both the longitudinal axis and the first transverse direction between the holding position where the holding surface of the slider is engaged with the counter holding surface of the plunger rod and the released position where the holding surface of the slider is disengaged with the counter holding surface of the plunger rod.

17. The sub-assembly of the medicament delivery device according to claim 15, wherein the inclined surface extends from a first end to a second end in the direction that is angled relative to both the longitudinal axis and to a plane transverse to the longitudinal axis.

18. The sub-assembly of the medicament delivery device according to claim 16, wherein the inclined surface faces towards the proximal end of the housing, and wherein the actuating surface faces towards the distal end of the housing.

19. The sub-assembly of the medicament delivery device according to claim 16, wherein the actuator comprises a body and a protrusion extending from a wall of the body, and wherein the actuating surface of the actuator is arranged on the protrusion.

20. The sub-assembly of the medicament delivery device according to claim 19, wherein the slider comprises a transverse surface extending in the direction of the longitudinal axis and facing in the second transverse direction, and wherein the protrusion of the actuator comprises a counter transverse surface that is configured to be adjacent to the transverse surface of the slider before the actuating surface contacts the first end of the inclined surface.

21. The sub-assembly of the medicament delivery device according to claim 20, wherein an interface formed between the holding surface of the slider and the counter holding surface of the plunger rod is a ramp surface.

22. The sub-assembly of the medicament delivery device according to claim 15, wherein the slider comprises a slider body extending along the longitudinal axis between a proximal end and a distal end.

23. The sub-assembly of the medicament delivery device according to claim 22, wherein the slider comprises a ledge extending from a surface of the body in the first transverse direction, and wherein the inclined surface defined by the ledge.

24. The sub-assembly of the medicament delivery device according to claim 15, wherein the plunger rod comprises an elongated plunger rod body extending along the longitudinal axis between a proximal end and a distal end, wherein the elongated plunger rod body of the plunger rod comprises an enlarged proximal part, wherein the enlarged proximal part has a diameter greater than a diameter of the rest of the body, and wherein the counter holding surface of the plunger rod is arranged on a surface of the enlarged portion of the body.

25. The sub-assembly of the medicament delivery device according to claim 15, wherein the counter holding surface of the plunger rod comprises a first counter holding surface and a second counter holding surface, and wherein the first counter holding surface is contiguous with and axially offset relative to the second counter holding surface.

26. The sub-assembly of the medicament delivery device according to claim 15, wherein the slider comprises a second inclined surface extending from the wall of the body in first transverse direction, and extending from a first end to a second end in a direction that is angled relative to both the longitudinal axis and to a plane transverse to the longitudinal axis, wherein the actuator comprises a second actuating surface, and wherein the second actuating surface is axially movable along the second inclined surface from the first end to the second end.

27. The sub-assembly of the medicament delivery device according to claim 15, wherein the housing comprises a housing body extending along a longitudinal axis between a proximal end and a distal end, and a flexible arm attached to the proximal end of the housing body.

28. The sub-assembly of the medicament delivery device according to claim 27, wherein the flexible arm extends from the housing body towards the longitudinal axis.
